(19) United States
(12) Patent Application Publication
(10) Pub. No.: US 2007/0014828 A1
(43) Pub. Date: Jan. 18, 2007

(54) Cross-linked nitric oxide-releasing polyamine coated substrates, compositions comprising same and method of making same

(75) Inventors: Anthony L. Fitzhugh, Frederick, MD (US); Peiwen Cheng, Santa Rosa, CA (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Government of the USA, represented by the Secretary, Dept. of Health and Human Services, Rockville, MD (US)

(21) Appl. No.: 11/477,642

(22) Filed: Jun. 29, 2006

Related U.S. Application Data
(63) Continuation of application No. 10/523,123.
(60) Provisional application No. 60/400,817, filed on Aug. 2, 2002.

(51) Int. Cl. A61L 33/00; A61F 2/02

(52) U.S. Cl. 424/423; 427/2.24

(57) ABSTRACT

The invention provides a method for preparing a nitric oxide-releasing medical device. The method includes contacting an amine-functionalized silane residue with a substrate, e.g., a metallic substrate, contacting the amine-functionalized silane residue with a cross-linking agent, contacting at least one nucleophilic residue with the cross-linked amine-functionalized silane residue, and contacting the nucleophilic residue with nitric oxide gas. The invention also provides a method of contacting the cross-linked amine-functionalized silane residue with at least one nitric oxide-releasing functional group. Furthermore, the invention provides a medical device for delivering nitric oxide in therapeutic a concentration, wherein the device comprises a substrate having nitric oxide bound thereto through diazeniumdiolated nucleophiles bonded to silane intermediates. The silane intermediates are bonded to the substrate and are amine-functionalized and cross-linked.

### FIELD OF THE INVENTION

This invention pertains to a cross-linked nitric oxide-releasing substrates, compositions comprising same and method of making same.

### BACKGROUND OF THE INVENTION

Nitric oxide (NO) is a simple diatomic molecule that plays a diverse and complex role in cellular physiology. It is known that NO is a powerful signaling compound and cytotoxic/cytostatic agent found in nearly every tissue of the human body, including endothelial cells, neural cells, and macrophages. NO has been implicated recently in a variety of bioregulatory processes, including normal physiological control of blood pressure, angiogenesis, and thrombosis, as well as neurotransmission, cancer, and infectious diseases. See, e.g., Moncada, “Nitric Oxide,” J Hypertens. Suppl. 12(10): S35-39 (1994); Moncada et al., “Nitric Oxide from L-Arginine: A Bioregulatory System,” Excerta Medica, International Congress Series 897 (Elsevier Science Publishers B.V.: Amsterdam, 1990); Marletta et al., “Unraveling the Biological Significance of Nitric Oxide,” Biofactors 2: 219-225 (1990); Ignarro, “Nitric Oxide. A Novel Signal Transduction Mechanism for Transcellular Communication,” Hypertension 16: 477-483 (1990); Hariawala et al., “Angiogenesis and the Heart: Therapeutic Implications,” J.R. Soc. Med. 90(6): 307-311 (1997); Granger et al., “Molecular and Cellular Basis of Myocardial Angiogenesis,” Cell. Mol. Biol. Res. 40(2): 81-85 (1994); Chiueh, “Neuroprotective Properties of Nitric Oxide,” Ann. N.Y. Acad. Sci. 890: 301-311 (1999); Wink et al., “The Role of Nitric Oxide Chemistry in Cancer Treatment,” Biochemistry (Moscow) 63(7): 802-807 (1998); Fang, F. C., “Perspectives Series: Host/Pathogen Interactions. Mechanisms of Nitric Oxide-Antimicrobial Activity,” J Clin. Invest. 99(12): 2818-25 (1997); and Fang, F. C., “Nitric Oxide and Infection,” (Kluwer Academic/Plenum Publishers: New York, 1999).

Glyceryl trinitrate and sodium nitroprusside are two examples of vasodilators that currently enjoy widespread clinical use and whose pharmacological actions result from their metabolic conversion in situ to NO-releasing species. See, e.g., Ignarro et al., J Pharmocol. Exp. Ther. 218: 739-749 (1981); Ignarro, Annu. Rev. Pharmacol. Toxicol. 30: 535-560 (1990); and Kruszyna et al., Chem. Res. Toxicol. 3: 71-76 (1990). In addition, other agents have been described in the literature which release NO spontaneously or following metabolic conversion of their parent or prodrug forms. See, e.g., Drago, ACS Adv. Chem. Ser. 36: 143-149 (1962); Longhi and Drago, Inorg. Chem. 2: 85 (1963); Schönafinger, “Heterocyclic NO prodrugs,” Farmaco 54(5): 316-320 (1999); Hou et al., “Current trends in the Development of Nitric Oxide Donors,” Curr. Pharm. Des. 5(6): 417-441 (1999); Muscara et al., “Nitric Oxide. V. Therapeutic Potential of Nitric Oxide Donors and Inhibitors,” Am. J. Physiol. 276(6, Pt. 1): G1313-1316 (1999); Maragos et al., “Complexes of NO with Nucleophiles as Agents for the Controlled Biological Release of Nitric Oxide. Vasorelaxant Effects,” J. Med. Chem. 34: 3242-3247 (1991); Fitzhugh et al., “Diazeniumdiolates: pro- and antioxidant applications of the ‘NONOates,’” Free Radic. Biol. Med. 28(10): 1463-1469 (2000); Saavedra et al., “Diazeniumdiolates (Formerly NONOates) in Cardiovascular Research and Potential Clinical Applications,” Nitric Oxide and the Cardiovascular System (Humana Press: Totowa, New Jersey, 2000); and Yamamoto et al., “Nitric oxide donors,” Proc. Soc. Exp. Biol. Med. 225(3): 200-206 (2000).

NO-donor compounds can exert powerfill tumoricidal and cytostatic effects. Such effects are attributable to NO's ability to inhibit mitochondrial respiration and DNA synthesis in certain cell lines. In addition to these bioregulatory properties, NO may arrest cell migration. These effects are apparently not limited to NO-donor compounds as macrophages can also sustain high levels of endogenous NO production via enzymatic mechanisms. Similar inhibitory effects have also been observed in other cells. See, e.g., Hibbs et al., “Nitric Oxide: A Cytotoxic Activated Macrophage Effector Molecule,” Biochem. and Biophys. Res. Comm. 157: 87-94 (1988); Stuehr et al., “Nitric Oxide. A Macrophage Product Responsible for Cytostasis and Respiratory Inhibition in Tumor Target Cells,” J Exp. Med. 169: 1543-1555 (1989); Zingarelli, et al., “Oxidation, Tyrosine Nitration and Cytostasis Induction in the Absence of Inducible Nitric Oxide Synthase,” Int. J. Mol. Med. 1(5): 787-795 (1998); Yamashita et al., “Nitric Oxide is an Effector Molecule in Inhibition of Tumor Cell Growth by rIFN-gamma-activated Rat Neutrophils,” Int. J Cancer 71(2): 223-230 (1997); Garg et al., “Nitric oxide-Generating Vasodilators Inhibit Mitogenesis and Proliferation of BALB/C 3T3 Fibroblasts by a Cyclic GMP-Independent Mechanisms,” Biochem. and Biophys. Res. Comm. 171: 474-479 (1990); and Sarkar et al., “Nitric Oxide Reversibly Inhibits the Migration of Cultured Vascular Smooth Muscle Cells,” Circ. Res. 78(2): 225-30 (1996).

Medical research is rapidly discovering a number of potential therapeutic applications for NO-releasing compounds/materials, particularly in the fields of vascular surgery and interventional cardiology. For example, fatty deposits may build up on the wall of an artery as plaque. Over time as additional material is added, the plaque thickens, dramatically narrowing the cross-sectional area of the vessel lumen in a process known as arteriosclerosis. Blood flow to the heart muscle is compromised resulting in symptoms ranging from intermittent chest pain to easy fatigability. In an effort to reduce such symptoms and improve blood flow, patients with this condition may opt to undergo a procedure known as coronary artery bypass grafting (CABG). In a typical CABG procedure, a portion of a vein is removed from the leg. Sections of the vein are then used to bypass the site(s) of plaque-induced coronary artery narrowing. CABG involves a major surgical procedure wherein the patient's chest is opened to facilitate the operation, as a result, it carries with it appreciable morbidity and mortality risks. However, bypassing the site(s) of greatest narrowing with a grafted vein substantially alleviates the chest pain and fatigue that are common in this condition while reducing the risk of acute arterial blockage. A less invasive and increasingly common procedure for treating plaque-narrowed coronary arteries is called percutaneous transluinal coronary angioplasty (PTCA) (also known as balloon angioplasty). In PTCA, a catheter is inserted into the femoral artery of the patient's leg and threaded through the circulatory system until the site of coronary vessel occlusion is reached. Once at the site, a balloon on the tip of the catheter is inflated which compresses the plaque against the wall of the vessel. The balloon is then deflated and the catheter removed. PTCA results in dramatic improvement in coronary blood flow as the cross-sectional area of the vessel lumen is increased substantially by this procedure. However, common complications of this procedure include thrombus formation at the site of PTCA-treatment, vessel rupture from overextension, or complete collapse of the vessel immediately following deflation of the balloon. These complications can lead to significant alterations in blood flow with resultant damage to the heart muscle.

To limit many of the problems associated with PTCA-treatment, cardiologists will frequently insert a small tubular device known as a stent. The stent serves as a permanent scaffold for maintaining vessel patency following deflation and removal of the balloon-tipped catheter from the artery. Since the stent is a permanent implant, its insertion can cause the vessel wall at the site of PTCA-injury to respond in a complex multi-factorial process known as restenosis. This process is initiated when thrombocytes (platelets) migrate to the injury site and release mitogens into the injured endothelium. Clot formation or thrombogenesis occurs as activated thrombocytes and fibrin begin to aggregate and adhere to the compressed plaque on the vessel wall. Mitogen secretion also causes the layers of vascular smooth muscle cells below the site of injury (neointima) to over proliferate, resulting in an appreciable thickening of the injured vessel wall. Within six months of PTCA-treatment roughly 30 to 50% of patients will exhibit significant or complete re-occlusion of the vessel.

Nitric oxide has recently been shown to dramatically reduce thrombocyte and fibrin aggregation/adhesion and smooth muscle cell hyperplasia while promoting endothelial cell growth (Cha et al., “Effects of Endothelial Cells and Mononuclear Leukocytes on Platelet Aggregation,” Haematologia (Budap) 30(2): 97-106 (2000); Lowson et al., “The Effect of Nitric Oxide on Platelets When Delivered to the Cardiopulmonary Bypass Circuit,” Anest. Analg. 89(6): 1360-1365 (1999); Riddel et al., “Nitric Oxide and Platelet Aggregation,” Vitam. Horm. 57: 25-48 (1999); Gries et al., “Inhaled Nitric Oxide Inhibits Human Platelet Aggregation, P-selectin expression, and Fibrinogen Binding, In Vitro and In Vivo,” Circulation 97(15): 1481-1487 (1998); and Lüscher, “Thrombocyte-vascular Wall Interaction and Coronary Heart Disease,” Schweiz ‘Med. Wochenschr’ 121(51-52): 1913-1922 (1991)). NO is one of several “drugs” under development by researchers as a potential treatment for the restenotic effects associated with intracoronary stent deployment. However, because the cascade of events leading to irreparable vessel damage can occur within seconds to minutes of stent deployment, it is essential that any anti-restenotic “drug” therapy be available at the instant of stent implantation. Also, it is widely thought that such therapy may need to continue for some time afterwards as the risk of thrombogenesis and restenosis persists until an endothelial lining has been restored at the site of injury.

In theory, one approach for treating such complications involves prophylactically supplying the PTCA-injury site with therapeutic levels of NO. This can be accomplished by stimulating the endogenous production of NO or using exogenous NO sources. Methods to regulate endogenous NO release have primarily focused on activation of enzymatic pathways with excess NO metabolic precursors like L-arginine and/or increasing the local expression of nitric oxide synthase (NOS) using gene therapy. U.S. Pat. Nos. 5,945,452, 5,891,459, and 5,428,070 describe the sustained NO elevation using orally administrated L-arginine and/or L-lysine while U.S. Pat. Nos. 5,268,465, 5,468,630, and 5,658,565 describe various gene therapy approaches. Other various gene therapy approaches have been described in the literature. See, e.g., Smith et al., “Gene Therapy for Restenosis,” Curr. Cardiol. Rep. 2(1): 13-23 (2000); Alexander et al., “Gene Transfer of Endothelial Nitric Oxide Synthase but not Cu/Zn Superoxide Dismutase restores Nitric Oxide Availability in the SHRSP,” Cardiovasc. Res. 47(3): 609-617 (2000); Channon et al., “Nitric Oxide Synthase in Atherosclerosis and Vascular Injury: Insights from Experimental Gene Therapy,” Arterioscler. Thromb. Vasc. Biol. 20(8): 1873-1881 (2000); Tanner et al., “Nitric Oxide Modulates Expression of Cell Cycle Regulatory Proteins: A Cytostatic Strategy for Inhibition of Human Vascular Smooth Muscle Cell Proliferation,” Circulation 101(16): 1982-1989 (2000); Kibbe et al., “Nitric Oxide Synthase Gene Therapy in Vascular Pathology,” Semin. Perinatol. 24(1): 51-54 (2000); Kibbe et al., “Inducible Nitric Oxide Synthase and Vascular Injury,” Cardiovasc. Res. 43(3): 650-657 (1999); Kibbe et al., “Nitric Oxide Synthase Gene Transfer to the Vessel Wall,” Curr. Opin. Nephrol. Hypertens. 8(1): 75-81 (1999); Vassalli et al., “Gene Therapy for Arterial Thrombosis,” Cardiovasc. Res. 35(3): 459-469 (1997); and Yla-Herttuala, “Vascular Gene Transfer,” Curr. Opin. Lipidol. 8(2): 72-76 (1997). However, these methods have not proved clinically effective in preventing restenosis. Similarly, regulating endogenously expressed NO using gene therapy techniques such as NOS vectors remains highly experimental. Also, there remain significant technical hurdles and safety concerns that must be overcome before site-specific NOS gene delivery will become a viable treatment modality.

The exogenous administration of gaseous nitric oxide is not feasible due to the highly toxic, short-lived, and relatively insoluble nature of NO in physiological buffers. As a result, the clinical use of gaseous NO is largely restricted to the treatment of neonates with conditions such as persistent pulmonary hypertension (Weinberger et al., “The Toxicology of Inhaled Nitric Oxide,” Toxicol. Sci. 59(1), 5-16 (2001); Kinsella et al., “Inhaled Nitric Oxide: Current and Future Uses in Neonates,” Semin. Perinatol. 24(6), 387-395 (2000); and Markewitz et al., “Inhaled Nitric Oxide in Adults with the Acute Respiratory Distress Syndrome,” Respir. Med. 94(11), 1023-1028 (2000)). Alternatively, however, the systemic delivery of exogenous NO with such prodrugs as nitroglycerin has long enjoyed widespread use in the medical management of angina pectoris or the “chest pain” associated with atherosclerotically narrowed coronary arteries. There are problems with the use of agents such as nitroglycerin. Because nitroglycerin requires a variety of enzymes and cofactors in order to release NO, repeated use of this agent over short intervals produces a diminishing therapeutic benefit. This phenomenon is called drug tolerance and results from the near or complete depletion of the enzymes/cofactors needed in the blood to efficiently convert nitroglycerin to a NO-releasing species. By contrast, if too much nitroglycerin is initially given to the patient, it can have devastating side effects including severe hypotelision and free radical cell damage.

Because of problems associated with the systemic delivery of NO, there has been a recent shift towards identifying agents/materials capable of directly releasing NO or other antirestenotic agents over a prolonged period directly at the site of PTCA-vascular injury. As a result, there exists a substantial need for a stent comprised of or coated with a material capable of continuously releasing NO from the instant of contact with a blood field to days or weeks following its deployment in a coronary artery. Such a device potentially represents an ideal means of treating the restenosis that frequently accompanies the implantation of a stent into a coronary artery. See, e.g., U.S. Pat. Nos. 6,087,479 and 5,650,447, U.S. Patent Application No. 2001/0000039, and PCT No. WO 00/02501, that detail prior art approaches to developing NO-releasing coatings for metallic stents and other medical devices.

Diazeniumdiolates comprise a diverse class of NO-releasing compounds/materials that are known to exhibit sufficient stability to be useful as therapeutics. Although discovered more than 100 years ago by Traube et al., Liebigs Ann. Chem. 300:81-128 (1898), the chemistry and properties of diazeniumdiolates have been extensively reinvestigated by Keefer and co-workers, as described in U.S. Pat. Nos. 4,954,526, 5,039,705, 5,155,137, 5,212,204, 5,250,550, 5,366,997, 5,405,919, 5,525,357, and 5,650,447, and in J. A. Hrabie et al., J. Org. Chem. 58: 1472-1476 (1993), and incorporated herein by reference.

Because many NO-releasing diazeniumdiolates have been prepared from amines, one potential approach for treating PTCA-associated restenosis is to coat the device with a suitably diazeniumdiolated amine-functionalized polymeric material. U.S. Pat. No. 5,405,919, for example, describes several biologically acceptable, amine-functionalized polyolefin-derived polymers. However, there are a number of problems associated with polyolefin-based coatings. They are prone to fractures as the coating is stressed during procedures such as stent expansion. Were such fractures to occur, it might cause particulate fragments from the coating to be released into the lumen of the overstretched vessel, ultimately lodging downstream in much narrower arteriolae and capillaries and compromising blood flow to those portions of the heart muscle that are supplied by the affected artery. Additionally, polyolefin-based and -coated medical devices tend to be more prone to the development of biofilms and device-related infections. These problems suggest that polyolefin-based materials may not be appropriate for uses in which permanent in situ implantation is desired. By contrast, metallic medical devices have repeatedly been shown to exhibit bio- and hemocompatibility properties that are superior to many polyolefin-based materials. See, Palmaz, “Review of Polymeric Graft Materials for Endovascular Applications,” J Vasc. Interv. Radiol. 9(1 Pt. 1): 7-13 (1998); Tepe et al., “Covered Stents for Prevention of Restenosis. Experimental and Clinical Results with Different Stent Designs,” Invest. Radiol. 31(4): 223-229 (1996); Fareed, “Current Trends in Antithrombotic Drug and Device Development,” Semnin. Thromb. Hemost. 22(Suppl. 1): 3-8 (1996); Bolz et al., “Coating of Cardiovascular Stents with a Semiconductor to Improve Their Hemocompatibility,” Tex. Heart Inst. J. 23(2): 162-166 (1996); De Scheerder et al., “Biocompatibility of Polymer-Coated Oversized Metallic Stents Implanted in Normal Porcine Coronary Arteries,” Atherosclerosis 114(1): 105-114 (1995); and Libby et al., “Ultrasmooth Plastic to Prevent Stent Clogging,” Gastrointest. Endosc. 40(3): 386-387 (1994). More recently, quite dramatic improvements in bio- and hemocompatibility have also been observed in medical devices coated with certain polymeric materials (e.g., silicone, hydrogel, heparin-, albumin-, phosphorylcholine-functionalized polymers and the like). See, e.g., Malik et al., “Phosphorylcholine-Coated Stents in Porcine Coronary Arteries. In Vivo Assessment of Biocompatibility,” J. Invasive Cardiol. 13(3): 193-201 (2001); Tsang et al., “Silicone-Covered Metal Stents: An In Vitro Evaluation for Bioflim Formation and Patency,” Dig. Dis. Sci. 44(9): 1780-1785 (1999); Kuiper et al., “Phosphorylcholine-coated Metallic Stents in Rabbit Illiac and Porcine Coronary Arteries,” Scand. Cardiovasc. J 32(5): 261-268 (1998); and McNair, “Using Hydrogel Polymers for Drug Delivery,” Med. Device Technol. 7(10): 16-22 (1996).

Beyond the type of material used to coat the medical device, methods for precisely dosing NO have not yet been perfected with any of the NO-releasing diazeniumdiolated compounds/materials that have been developed to date. When exposed to hydrogen ion (i.e., proton) donors such as, for example, water or physiological fluids, most diazeniumdiolates bearing unshielded and unprotected [(NO)NO]⁻ groups rapidly break down to produce a “burst” of NO. This initial surge or burst of NO is typically followed by a steady but diminishing rate of release until the entire NO content of the material has been exhausted. For most diazeniumdiolated compounds, such processes are complete within minutes to a few hours of the initial NO burst.

Accordingly, there remains a need for an NO-releasing medical device suitable for use in the treatment of various medical indications and which are compatible with the animal body, including the human body and internal organs, blood vessels, tissues and cells. Desirably such devices are capable of the sustained release of NO for periods lasting days to a few weeks or longer. The invention described herein provides for the preparation of such coated medical devices. These and other advantages of the present invention, as well as additional inventive features, will be apparent from the description of the invention provided below.

### BRIEF SUMMARY OF THE INVENTION

The invention provides a method for preparing a nitric oxide-releasing substrate. Specifically, the method includes contacting an amine-functionalized silane residue with a substrate, contacting the amine-functionalized silane residue with a cross-linking agent, and contacting at least one nitric oxide-releasing functional group with the cross-linked amine-functionalized silane residue.

The invention provides another method for preparing a nitric oxide-releasing substrate, the method including contacting an amine-functionalized silane residue with a substrate, contacting the amine-functionalized silane residue with a cross-linking agent, and contacting at least one nitric oxide-releasing nucleophilic residue with the cross-linked amine-functionalized silane residue. Nitric oxide gas is contacted with the nucleophilic residues on the substrate to form a nitric oxide-releasing functional group on the substrate.

It will be appreciated that in each of the methods discussed above that the method can be used to alter the surface of the substrate to impart to the surface the desired nitric oxide-releasing capability.

The invention further provides a medical device for delivering nitric oxide in therapeutic amounts. Specifically, the medical device of the invention includes a substrate to which the amine-functionalized silane residue can be bound, such as, for example, a metallic surface, and nitric oxide bound to the substrate through NO-releasing nucleophiles which are bonded to amine-functionalized and cross-linked silane intermediates.

The term “medical device” refers to any device, product, equipment or material having surfaces that contact tissue, blood, or other bodily fluids in the course of their use or operation, which fluids are found in or are subsequently used in patients or animals. Medical devices include, for example, extracorporeal devices for use in surgery, such as blood oxygenators, blood pumps, blood storage bags, blood collection tubes, blood filters including filtration media, tubing used to carry blood and the like which contact blood which is then returned to the patient or animal. Medical devices also include endoprostheses implanted in a human or animal body, such as stents, pacemaker, pacemaker leads, heart valves, pulse generator, cardiac defibrillator, cardioverter defibrillator, spinal stimulator, brain and nerve stimulator, introducer, chemical sensor, and the like, that are implanted in blood vessels or the heart. Medical devices also include devices for temporary intravascular use such as catheters, guide wires, amniocentesis and biopsy needles, cannulae, drainage tubes, shunts, sensors, transducers, probes and the like which are placed into the blood vessels, the heart, organs or tissues for purposes of monitoring, repair or treatment. Medical devices also include prostheses such as hips or knees as well as artificial hearts. Medical devices also include implants, specula, irrigators, nozzles, calipers, forceps, retractors, vascular grafts, personal hygiene items, absorbable and nonabsorbable sutures, wound dressings, and the like.

### DETAILED DESCRIPTION OF THE INVENTION

The invention provides medical devices which are capable of releasing nitric oxide-releasing when in use, but which are otherwise inert to nitric oxide release. In particular, NO-releasing functional groups are bound to a substrate that is coated with an amine-functionalized silane residue, more particularly a polysiloxane residue. Alternatively, nucleophllic residues are bound to the substrate, followed by diazeniumdiolation with nitric oxide. The nucleophilic residues may form part of the substrate, or are present as pendant groups attached to molecules and/or polymers covalently linked to the substrate. The term “bound” as used herein includes covalent bonds, ionic bonds, van der Waal forces, hydrogen bonding, electrostatic bonding, and all other methods for attaching nitric oxide to a substrate.

The term “diazeniumdiolation,” as used herein, refers to the process of contacting a nucleophile residue with NO gas to produce a nitric oxide-releasing nucleophile residue complex containing the [N(O)NO] subunit. Reaction of the amine-functionalized polysilane with NO can occur by any method known in the art. Diazeniumdiolation can occur either through the neat exposure to NO gas or by immersing the coated substrate in an organic solvent and then exposing the solution to NO. Typical organic solvents include, for example, acetonitrile, diethyl ether, tetrahydrofuran, dioxane or mixtures thereof. In the solvent system, the NO gas can be bubbled into the solvent containing the coated substrate or added under mild or elevated pressure using typical equipment and methods known in the art. Additionally, any temperature can be used so long as it allows for the formation of at least one nitric oxide-releasing diazeniumdiolate group.

One preferred embodiment of the invention provides a method for preparing a nitric oxide-releasing substrate. Specifically, the method includes: (a) contacting the amine-functionalized silane residue with a substrate; (b) contacting the amine-functionalized silane residue with a cross-linking agent; and (c) contacting at least one nitric oxide-releasing functional group with the cross-linked amine-functionalized silane residue.

The substrate can be any material capable of reacting with silanes. The substrate can be of any form, including a sheet, a fiber, a tube, a fabric, an amorphous solid, an aggregate, dust, or the like. Exemplary substrate materials include metal, glass, ceramic, plastic, rubber, natural fibrous materials, synthetic fibrous materials, or any combination thereof. Natural materials include cotton, silk, linen, hemp, wool, and the like. More preferably, the substrate is a metal, glass, ceramic, plastic or rubber substrate. Most preferably, the substrate is metal. Advantageously, the substrate comprises a biocompatible material.

Exemplary metal substrates include stainless steel, nickel, titanium, iron, tantalum, aluminum, copper, gold, silver, platinum, zinc, silicon, magnesium, tin, alloys, coatings containing any of the above and combinations of any of the above. Also included are such metal substrates as galvanized steel, hot dipped galvanized steel, electrogalvanized steel, annealed hot dipped galvanized steel and the like. Preferably, the metal substrate is stainless steel.

Exemplary glass substrates include soda lime glass, strontium glass, borosilicate glass, barium glass, glass-ceramics containing lanthanum, and combinations thereof.

Exemplary ceramic substrates include boron nitrides, silicon nitrides, aluminas, silicas, and combinations thereof.

Exemplary plastic substrates and synthetic fibrous materials include acrylics, acrylonitrile-butadiene-styrene, acetals, polyphenylene oxides, polyimides, polystyrene, polypropylene, polyethylene, polytetrafluoroethylene, polyvinylidene, polyethylenimine, polyesters, polyethers, polylactones, polyurethanes, polycarbonates, polyethylene terephthalate, as well as copolymers thereof and combinations thereof.

Exemplary rubber substrates include silicones, fluorosilicones, nitrile rubbers, silicone rubbers, fluorosilicone rubbers, polyisoprenes, sul-cured rubbers, isoprene-acrylonitrile rubbers, and combinations thereof. Silicones, fluorosilicones, polyurethanes, polycarbonates, polylactones, and mixtures or copolymers thereof are preferred plastic or rubber substrates because of their proven bio- and hemocompatability when in direct contact with tissue, blood, blood components, or bodily fluids.

Exemplary natural fibrous materials include cotton, linen, silk, hemp, wool, and combinations thereof.

Other exemplary substrates include those described in WO 00/63462, and are incorporated herein by reference, as well as combinations of the above-mentioned substrates.

Preferably, the substrate is cleaned according to procedures well known in the art prior to reaction with the silane reagent(s). To prepare the nitric oxide-releasing coated substrates of the invention, the substrate (e.g., stainless steel) is contacted with a composition containing an amine-functionalized silane compound or oligomer thereof.

The amine-functionalized silane compound is preferably hydrolyzed prior to contacting it with the substrate. More preferably, the amine-functionalized silane compound is dissolved, suspended, dispersed, or the like in a composition comprising a hydrolyzing reagent. Most preferably, the amine-functionalized silane compound is dissolved in a composition comprising a hydrolyzing reagent. The hydrolyzing reagent hydrolyzes the silane to form mono- and oligomeric silane. Advantageously, therefore, one or more silanes are dissolved in the hydrolyzing reagent, such as water, or solvent comprising the hydrolyzing reagent containing at least one molar equivalent of water to facilitate its hydrolysis such that oligomer formation is the predominant reaction. Preferable solvents for this transformation include those known in the art, such as, for example, methanol, ethanol, isopropanol, tetrahydrofaran, acetonitrile, and the like that are readily miscible with water. Optionally, however, the amine-functionalized silane compound can be mixed in a silicone gel containing at least one molar equivalent of water and applied to the substrate.

The amine-functionalized silane compositions or solutions are contacted with the substrate using methods known in the art including, for example, dipping, spraying, brushing, imbibing, and rolling. While not wishing to be bound to any particular theory, it is believed that after the amine-functionalized oligomeric silane composition is contacted with the substrate, functional groups (e.g., hydroxyls) on the surface of the substrate contact with the silane derivatives to form covalent bonds between silane and the substrate. Preferably, the silane-coated substrate is cured. Curing can occur at any temperature, pressure, or in the presence or absence of an inert gas/gas mixture, in the presence of absence of moisture, or an external energy source, such as heat or other radiation, e.g., gamma radiation, or mechanical energy, e.g., sonic energy, so long as the amine-functionalized polysilane layers formed during this step are not damaged, i.e., rendering them incapable of further coating cycles and/or diazeniumdiolation with NO. It is particularly preferred to cure the substrate under conditions that will preserve the nucleophile residue groups so that such groups are available for diazeniumdiolation. The number of such coating and curing cycles may be repeated to any desired level, so as to optimize the amount and period of NO released from the coated substrate.

The amine-functionalized silanes encompassed within the scope of the invention include any suitable silane compound capable of being bound to the substrate and that may be further derivatized with NO or nitric oxide-releasing functional groups to confer NO-releasing capabilities. Exemplary amine-functionalized silane compounds include those disclosed and described in, for example, U.S. Pat. Nos. 6,024,918, 6,040,058, 6,001,422, and 6,072,018, and PCT Nos. WO 99/37721 and WO 00/63462, and are incorporated herein by reference. Preferably, the amine-functionalized silane is any suitable compound, such as hydrolyzable silane compounds, having a reactive amino or polyaminoalkyl moiety attached to a di- or trialkoxysiloxane nucleus, including bis-aminosilanes having di- and trisubstituted silyl groups, wherein the hydrolyzable substituents include functionalities such as alkoxy, aryloxy, acyloxy, amine, chlorine and the like.

The aminosilanes and bis-aminosilanes can be described generally by the formulae shown below: 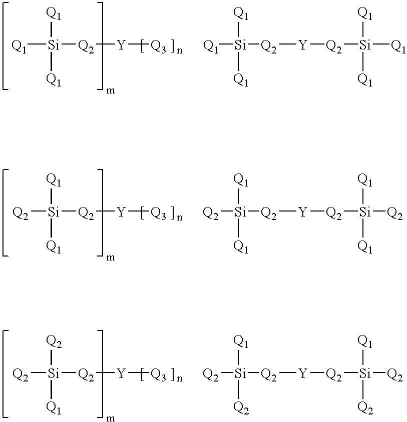
wherein m is either 1 or 2, n=(2−m), and Q₁ is an organofunctional moiety. Exemplary organofunctional moieties include alkoxy, aryloxy, acyloxy, amine, halo or derivatives thereof. The organofunctional moiety Q₁ can be unsubstituted or substituted C₁₋₂₄ aliphatic, unsubstituted or substituted C₃₋₁₂ olefinic, unsubstituted or substituted C₃₋₂₄ heterocycloalkyl, unsubstituted or substituted C₃₋₂₄ cycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or saccharides. The moiety Y is an amine-containing moiety. Exemplary amine-containing moieties include, for example, 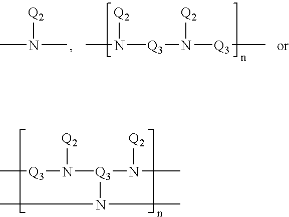
wherein n is an integer of 2-100. The moieties Q₂ and Q₃ can be the same or different and are organic or inorganic moieties. Exemplary organic or inorganic moieties Q₂ and Q₃ include nitric oxide-releasing functional groups as described herein, hydrogen, unsubstituted or substituted C₁₋₂₄ aliphatic, unsubstituted or substituted C₃₋₁₂ olefinic, unsubstituted or substituted C₃₋₂₄ cycloalkyl, unsubstituted or substituted C₃₋₂₄ heterocycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or mono- or polysaccharides. Preferred mono- and polysaccharides include ribose, glucose, deoxyribose, dextran, starch, glycogen, lactose, fucose, galactose, fructose, glucosamine, galactosamine, heparin, mannose, maltose, sucrose, sialic acid, cellulose, and combinations thereof.

All moieties of Q₁, Q₂, and Q₃, other than hydrogen, can be optionally substituted with 1 to 5 substituents, where the substituents can be the same or different. Exemplary substituents for Q₁₋₃ include nitro, halo, hydroxy, C₁₋₂₄ alkyl, C₁₋₂₄ alkoxy, amino, mono-C₁₋₂₄ alkylamino, di-C₁₋₂₄ alkylamino, cyano, phenyl and phenoxy. Also, Y can be optionally substituted. Exemplary substituents for Y include unsubstituted or substituted C₁₋₂₄ aliphatic polyamines, unsubstituted or substituted C₃₋₂₄ cycloallylamines, unsubstituted or substituted C₃₋₂₄ heterocycloalkylamines, unsubstituted, or substituted C₃₋₃₀ arylamines, such as unsubstituted or substituted phenyl amines, unsubstituted or substituted benzylamines, unsubstituted or substituted benzylamine carbonyls, unsubstituted or substituted phenylamine carbonyls, and combinations thereof.

Exemplary amine-functionalized silanes encompassed within the scope of the invention include 3-aminopropyltrimethoxysilane, 3-aminopropyltriethoxysilane, 3-aminopropyldimethoxysilane, N-(3-acryloxy-2-hydroxypropyl)-3-aminopropyltriethoxysilane, N-2-(aminoethyl)-3-aminopropyltris(2-ethyl-hexoxy)silane, 3-(m-aminophenoxy)propyltrimethoxysilane, 3-(1-aminopropoxy)-3,3-dimethyl-1-propenyltrimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylchlorosilane, bis(dimethylamino)methylmethoxysilane, bis(dimethylamino)phenylchlorosilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetriamine, trimethoxysilylpropylpentaethylenehexamine, triethoxysilyloctyldiethylenetriamine, triisopropoxysilylpentaethylenehexamine, 3-aminopropylmethyldiethoxysilane, 2-(perfluorooctyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-N′-[3-(dimethoxymethylsilyl)propyl]-1,2-ethanediamine, amine-functionalized polydimethylsiloxane copolymer (available from Dow Corning as “MDX4-4159”), and combinations thereof. The amine-functionalized silane compounds also include bis-aminosilanes such as, for example, bis-(trimetlioxysilylpropyl)amine, bis-(triethoxysilylpropyl)amine, bis-(triethoxysilylpropyl)ethylene diamine, N-[2-vinylbenzylamino)ethyl]-3-aminopropyltrimethoxysilane, aminoethylaminopropyltrimethoxysilane, trimethoxysilyl-modified polyethylenimine, methyldimethoxysilyl-modified polyethylenimine, and combinations thereof. Other exemplary amine-functionalized silanes include those disclosed and described in, for example, PCT Application No. WO 00/63462, and are incorporated by reference.

The amine-functionalized silanes can be used alone or in combination with one another. Additionally, the amine-functionalized silanes of the invention can be used as a mixture with other mono-, oligo-, or polymeric functionalized and nonfunctionalized silanes and silicones, such as, for example, 2-acetoxyethyltrichlorosilane, 2-acetoxyethyldimethylchlorosilane, acryloxypropylmethyldimethoxysilane, 3-acryloxypropyltrichlorosilane, 3-acryloxypropyltrimethoxysilane, adamantylethyltrichlorosilane, allyldimethylchlorosilane, allyltrichlorosilane, allyltriethoxysilane, allytrimetlioxysilane, amyltrichlorosilane, amyltriethoxysilane, amnyltrimethoxysilane, 5-(bicycloheptenyl)methyldichlorosilane, 5-(bicycloheptenyl)methyltriethoxysilane, 5-(bicycloheptenyl)methyltrimethoxysilane, 5-(bicycloheptenyl)dimethylmethoxysilane, 5-(bicycloheptenyl)methyldiethoxysilane, bis(3-cyanopropyl)dichlorosilane, bis(3-cyanopropyl)diethoxysilane, bis(3-cyanopropyl)dimethoxysilane, 1,6-bis(trimethoxysilyl)hexane, bis(trimethylsiloxy)methylsilane, bromomethyldimethylchlorosilane, bromomethyldimethylmethoxysilane, 3-bromopropyltrichlorosilane, 3-bromopropyltriethoxysilane, n-butyldimethylchlorosilane, n-butyldimethylmethoxysilane, tert-butyldimethylchlorosilane, ter-butyldimethylisoproplysilane, tert-butyldiphenylchlorosilane, tert-diphenylmethoxysilane, n-butylmethyldichlorosilane, n-butyldimethoxysilane, n-butyldiethoxysilane, n-butyldiisopropylsilane, n-butyltrimethoxysilane, (10-carbomethoxydecyl)dimethylchlorosilane, 2-(carbomethoxy)ethyltrinethoxysilane, 4-chlorobutyldimethylmethoxysilane, 4-chlorobutyldimethylethoxysilane, 2-chloroethylmethyldiisopropylsilane, 2-chloroethyltriethoxysilane, chloromethyldimethylethoxysilane, p-(chloromethyl)phenyltriethoxysilane, p-(chloromethyl)phenyltrimethoxysilane, chloromethyltriethoxysilane, chlorophenyltrimethoxysilane, 3-chloropropylmethyldimethoxysilane, 3-chloropropyltriethoxysilane, 2-(4-chlorosulfonylphenyl)ethyltrichlorosilane, 2-cyanoethylmethyltrimethoxysilane, (cyanomethylphenethyl)triethoxysilane, 3-cyanopropyldimethyldiisopropylsilane, 2-(3-cyclohexenyl)ethyl]trimethoxysilane, cyclohexydiethoxymethylsilane, cyclopentyltrimethoxysilane, di-t-butoxydiacetoxysilane, di-n-butyldimethoxysilane, dicyclopentyldimethoxysilane, diethyldiethoxysilane, diethyldimethoxysilane, diethyldibutoxysilane, diethylphophatoethyltriethoxysilane, diethyl(triethoxysilylpropyl)malonate, di-n-hexyldimnethoxysilane, diisopropyldichlorosilane, diisopropyldimethoxysilane, dimethyldiacetoxysilane, dimethyldimethoxysilane, 2,3-dimethylpropyldimethylethoxysilane, dimethylethoxysilane, dimethylmethoxychlorosilane, diimethyl-n-octadecylchlorsilane, N,N-dimethyltriethylsilylamine, 1,3-diemethyltetramethoxydisoloxane, diphenylchlorosilane, diphenyldiacetoxysilane, diphenydiethoxysilane, diphenyldifluorosilane, diphenyldimethoxysilane, diphenylmethylchlorosilane, diphenylmethylethoxysilane, 2-(diphenylphosphino)ethyltriethoxysilane, divinylethoxysilane, divinyldichlorosilane, n-docosylmethyldichlorosilane, n-dodecyltriethoxysilane, 2-(3,4-epoxycyclohexyl)ethyltrimethoxysilane, ethyldimethylchlorosilane, ethyltriacetoxysilane, ethyltriethoxysilane, ethyltrimethoxysilane, 3-glycidoxypropyldimethylethoxysilane, (3-glycidoxypropyl)methyldimethoxysilane, 3-glycidoxypropyltrimnethoxysilane, (3-heptafluoroisopropoxy)propylmethyldichlorosilane, n-heptylmethyldichlorosilane, n-heptylmethyldimethoxysilane, n-hexadecyltrichlorosilane, n-hexadecyltriethoxysilane, 6-hex-1-enyltrichlorosilane, 5-hexenyltrimethoxysilane, n-hexylmethyldichlorosilane, n-hexyltrichlorosilane, n-hexytriethoxysilane, n-hexyltnethoxysilane, 3-iodopropyltriethoxysilane, 3-iodopropyltrimethoxysilane, isobutyldimethylchlorosilane, isobutylmethyldichlorosilane, isobutyltrimethoxysilane, isobutyltriethoxysilane, 3-isocyanatopropyldimethylchlorosilanie, isocyanatopropyldimethylmethoxysilane, 3-isocyanatopropyltriethoxysilane, isooctyltrichlorsilane, isooctyltriethoxysilane, isopropyldimethylchlorosilane, 3-mercaptopropyh-nethyldimethoxysilane, 3-mercaptopropyltrimethoxysilane, 3-mercaptopropyltriethoxysilane, 3-methacryloxypropylnethyldiethoxysilane, 3-methacryloxypropyl-methyldirnethoxysilane, 3-methacryloxypropyltrimethoxysilane, 3-(4-methoxyphenyl)propyltrichlorosilane, 3-(4-methoxyphenyl)propyltrimethoxysilane, methylcyclohexyldichlorosilane, methylcyclohexyldiethoxysilane, methyldiacetoxysilane, methyldichlorosilane, methyldiethoxysilane, methyldimethoxysilane, methyldodecyldichlorosilane, methyldodecyldiethoxysilane, methylisopropyldichlorosilane, methyl-n-octadecyldtimethoxysilane, methyl-n-octyldichlorosilane, (p-methylphenethyl)methyldichlorosilane, methyl(2-phenethyl)dimethoxysilane, methylphenyldiisopropoxysilane, methylphenyldiethoxysilane, methylphenyldimethoxysilane, methyl-n-propyldimethoxysilane, methyltriacetoxysilane, methyltriethoxysilane, neophylmethyldiethoxysilane, n-octadecyldimethylmethoxysilane, n-octadecyltriethoxysilane, n-octadecyltrimethoxysilane, 7-oct-1-enylmethylchlorosilane, 7-oct-enyltrimethoxysilane, n-octyldiisopropylchlorosilane, n-octyldimethylchlorosilane, n-octylmethyldimethoxysilane, n-octyltriethoxysilane, 1,1,1,3,3-pentamiethyl-3-acetoxydisiloxane, phenethyldimethylchlorosilane, phenethyldimethylmethoxysilane, phenethyltriethoxysilane, phenyl(3-chloropropyl)dichlorosilane, phenyldimethylacetoxysilane, phenyldimethylethoxysilane, phenylmethylvinylchlorosilane, (3-phenylpropyl)dimethylchlorosilane, phenyltriethoxysilane, phenyltriethoxysilane, phthalocyanatodimethoxysilane, n-propyldimethylchlorosilane, n-propyltrimethoxysilane, styrylethyltrimethoxysilane, tetra-n-butoxysilane, tetraethoxysilane, tetramethoxysilane, tetraproproxysilane, (tridecafluoro-1,1 ,2,2,-tretrahydrooctyl)-1-trimethoxysilane, triethoxysilane, triethoxysilylpropylethyl carbamate, triethylacetoxysilane, triethylethoxysilane, (3,3,3-trifluoropropyl)dimethylchlorosilane, (3,3,3-trifluoropropyl)methyldimethoxysilane, (3,3,3-trifluoropropyl)triethoxysilane, trisopropylchlorosilane, trimethoxysilane, 1-trimethoxysilyl-2-(p,m-chloromethyl) phenyletlane, trimethylethoxysilane, 2-(trimethylsiloxy)ethyl methacrylate, p-trimethylsiloxynitrobenzene, o-trimethylsilylacetate, triphenylethoxysilane, n-undeceyltriethoxysilane, vinyldimethylethoxysilane, vinyltriacetoxysilane, vinyltrirnethoxysilane, and combinations thereof. Optionally, substrates can be alternatively or successively coated with amine-functionalized and functionalized/nonfunctionalized silanes and silicones. Additional functionalized and nonfunctionalized silanes and silicones encompassed within the scope of the invention include those disclosed and described in, for example, United Chemical Technologies, Inc. Catalog CD (1999-2000), and are incorporated herein by reference.

The nitric oxide-releasing functional group is any suitable group capable of releasing NO. The nitric oxide-releasing functional group is preferably a diazernumdiolated nitric oxide-releasing/nucleophile residue, i.e., a complex of nitric oxide and a nucleophile, most preferably a nitric oxide/nucleophile residue complex which contains the anionic moiety X [symbol] N(O)NO]⁻, X [symbol] N(O)NR [symbol] R or X—NO, where X is any suitable nucleophile residue. Preferably, nitric oxide-releasing functional groups of the invention are formed according to the following formula
X⁻+2NO→X [symbol] N(O)NO]⁻

The nucleophile residue is most preferably that of a primary amine (e.g., X=(CH₃)₂CHNH, as in (CH₃)₂CHNHN[N(O)NO]Na), a secondary amine (e.g., X=(CH₃CH₂)₂N, as in (CH₃CH₂)₂N[N(O)NO]Na), a polyamine (e.g., X=spermine, as in the zwitterions H₂N(CH₂)₃NH₂⁺(CH₂)₄N[N(O)NO]⁻(CH₂)₃NH₂, X=(ethylamino)ethyl in the zwitterion CH₃CH₂N[N(O)NO]⁻CH₂CH₂NH₃³⁰ , X=3-(n-propylamino)propylamine, as in the zwitterion CH₃CH₂CH₂N[N(O)NO]⁻CH₂CH₂CH₂NH₃⁺), oxide (i.e., X=O⁻, as in Na₂O[N(O)NO]), or derivatives thereof. Such nitric oxide/nucleophile residue complexes are stable as solids and are capable of releasing nitric oxide in a biologically useful form at a predictable rate. Most preferably, the nitric oxide/nucleophile residue complexes of the present invention are formed from a hydrolyzable amine-functionalized organosilane moiety. Suitable nitric oxide/amine-functionalized organosilanes include those described herein, wherein Q₂ is [N(O)NO]⁻ Q₂ or Q₃ is [N(O)NO [symbol] X; optionally, Q₂ and Q₃ are the same or different and are hydrogen, unsubstituted or substituted C₁₋₂₄ aliphatic, unsubstituted or substituted C₃₋₁₂ olefinic, unsubstituted or substituted C₃₋₂₄ cycloalkyl, unsubstituted or substituted C₃₋₂₄ heterocycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or saccharides. Preferred saccharides include ribose, glucose, deoxyribose, dextran, starch, glycogen, lactose, facose, galactose, fructose, glucosamine, galactosamine, heparin, mannose, maltose, sucrose, sialic acid and cellulose.

Other suitable nitric oxide/nucleophile residue complexes that can provide the NO-releasing functional group are well known in the art and include, for example, those described in U.S. Pat. Nos. 4,954,526, 5,039,705, 5,155,137, 5,121,204, 5,250,550, 5,366,997, 5,405,919, 5,525,357 and 5,650,447 to Keefer et al. and in Hirabie et al., J. Org. Chem. 58: 1472-1476 (1993), and are incorporated herein by reference.

Exemplary nitric oxide/nucleophile residue complexes that can provide the NO-releasing functional group include those having the following formulas: 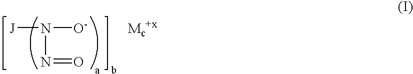
wherein J is an organic or inorganic moiety, including, for example, a moiety which is not linked to the nitrogen of the N₂O₂⁻ group through nitrogen atom, M^{+x} is a pharmaceutically acceptable cation, where x is the valence of the cation, a is 1 or 2, and b and c are the smallest integers that result in a neutral compound, preferably such that the compound is not a salt of alanosine or dopastin, as described in U.S. Pat. No. 5,212,204, and are incorporated herein by reference; 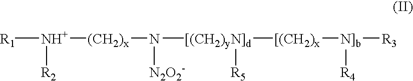
wherein b and d are the same or different and may be zero or one, R₁ , R₂, R₃, R₄, and R₅ are the same or different and may be hydrogen, C₃₋₈ cycloalkyl, C₁₋₁₂ straight or branched chain alkyl, benzyl, benzoyl, phthaloyl, acetyl, trifluoroacetyl, p-toluyl, t-butoxycarbonyl, or 2,2,2-trichloro-t-butoxycarbonyl, and x, y, and z are the same or different and are integers from 2 to 12, as described in U.S. Pat. No. 5,155,137, and are incorporated herein by reference; 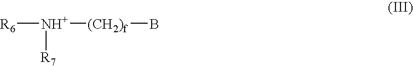
wherein B is 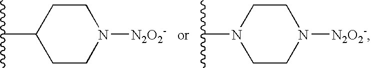
R₆ and R₇ are the same or different and are hydrogen, C₃₋₈ cycloalkyl, C₁₋₁₂ linear alkyl, or C₃₋₁₂ branched alkyl, benzyl, benzoyl, phthaloyl, acetyl, trifluoroacetyl, p-toluyl, t-butoxycarbonyl, or 2,2,2-trichloro-t-butoxycarbonyl, f is an integer from 0 to 12, with the proviso that when B is the substituted piperazine moiety 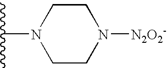
and f is an integer from 2 to 12, as described in U.S. Pat. No. 5,155,137, and are incorporated herein by reference; 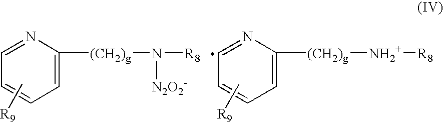
wherein R₈ is hydrogen, C₃₋₈ cycloalkyl, C₁₋₁₂ linear alkyl, C₃₋₁₂ branched alkyl, benzyl, benzoyl, phthaloyl, acetyl, trifluoroacetyl, p-toluyl, t-butoxycarbonyl, or 2,2,2-tri-chloro-t-butoxycarbonyl, R₉ is hydrogen or a C₁₋₁₂ linear alkyl, C₃₋₁₂ branched alkyl, and g is 2 to 6, as described in U.S. Pat. No. 5,250,550, and are incorporated herein by reference; 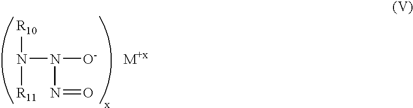
wherein R₁₀ and R₁₁ are independently selected from the group consisting of a linear C₁₋₁₂ arlyl or C₃₋₁₂ branched alkyl group and a benzyl group, preferably such that no branch occurs on the alpha carbon atom, or else R₁₀ and R₁₁, together with the nitrogen atom to which they are bonded, to form a heterocyclic group, preferably a pyrrolidino, piperidino, piperazino or morpholino group, M^{+x} is a pharmaceutically acceptable cation, and x is an integer from 1 to 10, as described in U.S. Pat. Nos. 5,039,705, 5,208,233 and 5,731,305, and are incorporated herein by reference;
K[(M)^{x′}_{x(L)}_{y}(R₁₂R₁₃N—N₂O₂)_{z}] (VI)
wherein M is a pharmaceutically acceptable metal, or, where x is at least two, a mixture of two different pharmaceutically acceptable metals, L is a ligand different from (R₁₂R₁₃N—N₂O₂) and is bound to at least one metal, R₁₂ and R₁₃ are each organic moieties and may be the same or different, x is an integer of from 1 to 10, x′ is the formal oxidation state of the metal M, and is an integer of from 1 to 6, y is an integer of from 1 to 18, and where y is at least 2, the ligands L may be the same or different, z is an integer of from 1 to 20, and K is a pharmaceutically acceptable counterion to render the compound neutral to the extent necessary, as described in U.S. Pat. No. 5,389,675, and are incorporated herein by reference;
[R₁₄N(H)N(NO)O⁻]_{y}X (VH)
wherein R₁₄ is C₂₋₈ alky, phenyl, benzyl, or C₃₋₈ cycloalkyl, any of which R₁₄ groups may be substituted by 1 to 3 substituents, which are the same or different, selected from the group consisting of halo, hydroxy, C₁₋₈ alkoxy, —NH₂, —C(O)NH₂, —CH(O), —C(O)OH, and —NO₂, X is a pharmaceutically acceptable cation, a pharmaceutically acceptable metal center, or a pharmaceutically acceptable organic group selected from the group consisting of C₁₋₈ alkyl, —C(O)CH₃, and —C(O)NH₂, and y is one to three, consistent with the valence of X, as described in U.S. Pat. No. 4,954,526, and are incorporated herein by reference; and 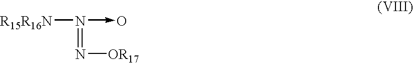
wherein R₁₅ and R₁₆ are independently chosen from C₁₋₁₂ linear alkyl, C₁₋₁₂ alkoxy or acyloxy substituted straight chain alkyl, C₁₋₁₂ hydroxy- or halo-substituted straight chain alkyl, C₃₋₁₂ branched chain alklyl, C₃₋₁₂ hydroxy-, halo-, alkoxy-, or acyloxy-substituted branched chain alkyl, C₃₋₁₂ linear alkenyl, and C₃₋₁₂ branched alkenyl which are unsubstituted or substituted with hydroxy, alkoxy, acyloxy, halo or benzyl, or R₁₅ and R₁₆, together with the nitrogen atom to which they are bonded, form a heterocyclic group, preferably a pyrrolidino, piperidino, piperazino or morpholino group, and R₁₇ is a group selected from C₁₋₁₂ linear and C₃₋₁₂ branched alkyl which are unsubstituted or substituted by hydroxy, halo, acyloxy or alkoxy, C₂₋₁₂ linear or C₃₋₁₂ branched alkenyl which are unsubstituted or substituted by halo, alkoxy, acyloxy or hydroxy, C₁₋₁₂ unsubstituted or substituted acyl, sulfonyl and carboxamido; or R₁₇ is a group of the formula —(CH₂)ₙ—ON=N(O)NR₁₅R₁₆, wherein n is an integer of 2-8, and R₁₅ and R₁₆ are as described above. Preferably R₁₅, R₁₆, and R₁₇ do not contain a halo or a hydroxy substituent alpha to a heteroatom, as described in U.S. Pat. No. 5,366,997, and are incorporated herein by reference.

Preferably, the nitric oxide-releasing functional group is at least one compound consisting of an O²-protected monodiazeniumdiolate of piperazine, such as the O²-glycosylated or methoxymethyl-protected monodiazeniumdiolate of piperazine. Another preferred nitric oxide-releasing functional group is a 1-[(2-carboxylato)pyrrolidin-1-yl]diazen-1-ium-1,2-diolate because the metabolite of the nitric oxide-releasing functional group is proline, an amino acid.

Other preferred nitric oxide/nucleophile residue complexes that can provide the NO-releasing functional group include O²-arylated and O²-glycosylated diazeniumdiolates, such as those described in the international patent application PCT/US97/17267 (filed Sep. 26, 1997), and are incorporated herein by reference. For example, a preferred O²-aryl substituted diazeniumdiolate has the following formula 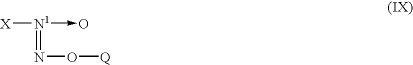
wherein X is selected from the group consisting of an amino, a polyamino, a C₁₋₂₄ aliphatic, a C₃₋₃₀ aryl, a C₃₋₃₀ nonaromatic cyclic, and an oxime, and Q is an optionally substituted aryl or heteroaryl group selected from the group consisting of an acridinyl, an anthracenyl, a benzyl, a benzofuranyl, a benzothiophenyl, a benzoxazolyl, a benzopyrazolyl, a benzothiazolyl, a carbazolyl, a chlorophyllyl, a cinnolinyl, a furanyl, an imidazolyl, an indolyl, an isobenzofuranyl, an isoindoleyl, an isoxazolyl, an isothiazolyl, an isoquinolinyl, a naphthalenyl, an oxazolyl, a phenanthrenyl, a phenanthridinyl, a phenothiazinyl, a phenoxazinyl, a phthalimidyl, a phthalazinyl, a phthalocyaninyl, a porphinyl, a pteridinyl, a purinyl, a pyrazinyl, a pyrazolyl, a pyridazinyl, a pyridinyl, a pyrimidinyl, a pyrrolyl, a quinolizium ion, a quinolinyl, a quinoxalinyl, a quinazolinyl, a sydnonyl, a tetrazolyl, a thiazolyl, a thiophenyl, a thyroxinyl, a triazinyl, and a triazolyl, wherein an atom of the ring of the aryl group is bonded to the O²-oxygen.

With respect to O²-glycosylated diazeniumdiolates, a preferred embodiment includes an O²-glycosylated 1-substituted diazen-1-ium-1,2-diolate of Formula IX. Preferably, X is selected from the group consisting of an amino, a polyamino, a C₁₋₂₄ aliphatic, a C₃₋₃₀ aryl and a C₃₋₃₀ non-aromatic cyclic, and Q is a saccharide. Optionally, Q is a protecting group, such as those known in the art (See, e.g., Greene et al., “Protecting Groups In Organic Synthesis,” J. Wiley & Sons: New York, 1999, and are incorporated herein by reference). Most preferably, the O²-substituted diazeniumdiolate includes an O²-substituted 1-[(2-carboxylato)pyrrolidin-1-yl]diazen-1-ium-1,2-diolate.

Other preferred nitric oxide/nucleophile residue complexes that can provide the NO-releasing functional group include enamine- and amidine-derived diazeniumdiolates, such as those described in the international patent publication No. WO 99/01427 (PCT/US98/13723), and are incorporated herein by reference.

The nitric oxide-releasing functional group may also be that of a polymer, e.g., a nitric oxide-releasing/nucleophile complex bound to a polymer such as those described in, for example, U.S. Pat. Nos. 5,405,919, 5,525,357, 5,632,981, 5,650,447, 5,676,963, 5,691,423, and 5,718,892, and are incorporated herein by reference. By “bound to a polymer” it is meant that the nitric oxide-releasing/nucleophile complex, such as those described by Formulae I-IX is associated with, part of, incorporated with, or contained within the polymer matrix physically or chemically. Physical association or bonding of the nitric oxide-releasing/nucleophile complex to the polymer may be achieved by co-precipitation of the polymer with the nitric oxide-releasing/nucleophile complex as well as by covalent bonding of the complex to the polymer. Chemical bonding of the nitric oxide-releasing/nucleophile complex to the polymer may be by, for example, covalent bonding of the nucleophile residue moiety of the nitric oxide-releasing/nucleophile complex to the polymer such that the nucleophile to which the NONO group is attached forms part of the polymer itself, i.e., is in the polymer backbone, or is attached to groups pendant to the polymer backbone. The manner in which the nitric oxide-releasing/nucleophile complex is associated, part of, or incorporated with or contained within, i.e., “bound” to the polymer, is inconsequential to the invention and all means of association, incorporation or bonding are contemplated herein. Preferably the nitric oxide-releasing/nucleophile complex is covalently bound to the polymer.

The nucleophile residue is preferably an amine-derived residue, e.g., primary or secondary amines, such as those described herein. The amine-derived nucleophile residue(s) is preferably a diethylenetriamine, pentaethylenehexamine, high molecular weight linear/branched polyethylenimines, polyamine-functionalized divinylbenzene, piperazine, or any combination thereof.

It has been found that substrates coated with amine-functionalized silanes in accordance with the invention were found to be sufficiently stable to (i) allow for diazeniumdiolation with NO and (ii) spontaneously release NO under physiological conditions. These unexpected results permit the development of medical devices, such as those described herein that are capable of sustained NO-release in accordance with the teachings of the invention.

The substrates can be converted into diazeniumdiolates once they have been provided with an amine-functionalized polysilane coating in accordance with the teachings of the invention. Briefly, the nitric oxide-releasing substrates of the invention are formed by contacting the previously processed substrates (cross-linked amine-functionalized silane-coated substrate) with nitric oxide or a nitric oxide-releasing functional group. Alternatively, the substrates can be converted into diazeniumndiolates once they have been provided with a nucleophile residue by contacting the nucleophile residue with NO gas either neat or, preferably, in a suitable solvent or solvent mixture.

Combinations of direct diazeniumdiolation and bonding of nitric oxide-releasing functional group are also within the scope of the invention.

In a preferred embodiment of the invention, the amine-functionalized silane compound is contacted with a cross-linking agent. It has been discovered that cross-linking the amine-functionalized silane compounds limits swelling when the silane-modified substrate is subjected to an aqueous solution, such as, for example, physiological fluids. Inhibiting or preventing swelling preserves the integrity of the NO-loaded substrate and prevents premature NO release. Avoiding rapid swelling of the coating greatly prolongs the rate at which water molecules are able to liberate the nitric oxide from the diazeniumdiolated amine-functionalized substrates. By contrast, the swelling that occurs in non-cross-linked NO-releasing coated surfaces permits water to quickly enter the interior of the amine-functionalized silane compound and contact with sequestered nitric oxide-releasing functional groups, thus liberating NO at a substantially increased rate. Moreover, as the functionalized, non-cross-linked polysilane coating swells, large channels are created that allow the liberated NO molecules to escape unhindered until the supply of releasable nitric oxide is substantially exhausted.

It is further believed that protic solvents (e.g., water) protonate the amine groups in the vicinity of the NO-releasing groups within the nucleophilic residues. These protonated amine groups may exert electrostatic repulsive effects, which inhibit protic attack on the NO-releasing groups, thus further sustaining the amount of NO released over time. See, e.g., Hrabie et al., J. Org. Chem. 58: 1472-1476 (1993), and incorporated herein by reference. The degree of cross-linking may be at any desired level, so as to optimize the time period of NO release.

The cross-linking agent can be any suitable homo- or heterobi- or homo- or heteromultifunctional compound. Typical suitable bi- or multifunctional cross-linking agents include, for example, dihalogenated alkyl, dihalogenated aryl groups, phenyl azides, maleimides, imidoesters, vinylsulfones, N-hydroxysuccinimide esters, haloacetyls, and hydroxymethyl phosphines. The cross-linking agents may be further substituted with 1 to 3 additional substituents. Preferably, these additional substituents consist of an alkyl, a cycloalkyl, hydroxyl, nitro, a halogen, or cyano. Preferred cross-linking agents are 1,4-dibromoetdune, 1,5-difluoro-2,4-dinitrobenzene, 1,4-bis-maleimidobutane, 1,4-bismaleimidyl-2,3-dihydoxybutane, bis-maleimdohexane, 1,11-bis-maelimidotetraethyleneglycol, bis[2-(succinimidyloxycarbonylethyl]sulfone, bis-[sulfosuccinimidyl]suberate, dimethyl adipimidate-2 HCl, dimethyl pimelimidate-2 HCl, disuccinimidyl glutarate, disuccinimidyl suberate, disuccinimidyl tartrate, ethylene glycol bis[succinimidylsuccinate], N-[p-maleimidophenyl]isocyanate, succinimidyl 3-[bromoacetamido]propionate, N-succinimidyl iodoacetate, bis[2-sulfosuccinimidooxycarbonyloxy)-ethyl]sulfone, disulfosuccinimidyl aminotriacetate, β-[tris(hydroxymethyl)phosphino]-propionic acid, and tris-[2-maleimidoethyl]amine. See, e.g. Pierce Chernical Company Catalog (2001-2002) (pgs. 294-343), and incorporated herein by reference.

Another embodiment of this invention includes a method for preparing a nitric oxide-releasing substrate, where the method includes: (a) contacting the amine-functionalized silane residue with a substrate; (b) contacting the amine-functionalized silane residue with a cross-linking agent; (c) contacting at least one nucleophilic residue with the cross-linked amine-functionalized silane residue; and (d) contacting the nucleophilic residue with nitric oxide gas.

In order to add a higher degree of cross-linking and therefore decreased water permeability, the method can further comprise after step (c), cross-linking the nucleophilic residue with a cross-linking agent followed by contacting at least one additional nucleophilic residue or, optionally, a nitric oxide-releasing functional group, with the cross-linked nucleophilic residue. The same type of cross-linking agent as described herein may be used to cross-link the nucleophilic residues to any degree. After reaching the desired level of cross-linking, additional nucleoplilic residues may be bound to the cross-linked nucleophilic residues to create reactive sites for diazeniumdiolation with NO gas. The preferred additional nucleophilic residues are those as described herein.

It is believed that the high degree of cross-linking forms a “lattice” or “matrix” structure that may residually trap NO within the lattice or matrix which, upon exposure to physiological solutions, release the trapped NO for a sustained period of time. In that regard, non- or weakly-nucleophilic residues of X are also envisioned to be within the scope of the present invention such that, when cross-linked with a suitable cross-linking agent, the residues form a chemical lattice or matrix serving to trap NO until exposure to physiological conditions.

If desired, before diazeniumdiolation with NO gas, the substrate having the cross-linked amine-functionalized polysilane residue can be treated with a bio- or hemocompatible topcoat. The biocompatible topcoat is any suitable lubricious hydrogel. Suitable lubricious hydrogels include, for example, hydrophilic silicones, homo- and heteropolyethers, polyols, polyureas, polylactones, albumin-, heparin-, and phosphorylcholine-functionalized polymers, or any combination thereof.

Another preferred embodiment of the invention is forming a hydrophobic topcoat on the substrate having the cross-linked amine-functionalized silane compound(s). Suitable hydrophobic topcoats include, for example, parylenes, polysiloxanes, and silicones functionalized with nonpolar substituents.

In yet another embodiment of this invention provides a medical device for delivering nitric oxide in therapeutic concentrations for a sustained period of time. The device includes a substrate having nitric oxide releasably bound thereto in the form of diazeniumdiolated nucleophilic residues. The polysilane intermediates are bonded to the substrate and are amine-functionalized and cross-linked.

The resulting diazeniumdiolated medical devices made in accordance with the invention can be tested to determine the concentration and duration of NO release upon exposure to physiological conditions by methods known in the art (e.g., immersion in phosphate buffered saline, pH 7.4 at 37° C.). Nitric oxide gas is preferably detected and quantified using the chemiluminescence methods as described in Keefer et al., “NONOates (1-Substituted Diazen-1-ium-1, 2 diolates) as Nitric Oxide Donors: Convenient Nitric Oxide Dosage Forms,” Methods in Enzymology 28: 281-293 (1996), and incorporated herein by reference.

The NO-releasing substrates of the invention have been found to generate between about 1,000 to about 40,000 pmoles per square millimeter (mm²) of coated substrate, more particularly between about 2,000 to about 35,000 pmoles per square millimeter (mm²), more particularly between about 5,000 to about 20,000 pmoles per square millimeter (mm²), and even more particularly between about 8,000 to about 13,000 pmoles per square millimeter (mm²). However, both the yield and duration of NO can be readily increased by coating the substrates with additional layers of the amine-functionalized polysilanes per the teachings of the invention. Moreover, the NO-releasing substrates of the invention can continually release NO for periods of hours to weeks or even longer. These findings far exceed those of any previously reported amine-functionalized polysilane coating in terms of the amounts or duration of NO released.

The cross-linked substrates of the invention provide localized release of nitric oxide under physiological conditions. The localized release or localized sustained release of NO provides in situ cytostatic, antithrombogenic, vasodilatory, antiproliferative, and other pharmacological effects. The NO-releasing substrates of the invention are thromboresistant when in contact with blood and are capable of inhibiting arterial restenosis as well promoting angiogeniesis. Accordingly, when used alone, as a coating on, or in combination with, other substances (e.g., stainless steel, glass, silicone rubber, plastics, natural fibrous materials, etc.) many uses are contemplated.

The NO-releasing substrates of the invention can be used to treat or prevent a wide range of conditions including, for example, ischemic heart disease, restenosis, cancer, hypertension, infectious diseases, and sexual dysfunction. Commercial applications include, for example, the preparation of coated NO-releasing medical devices, as described herein, including stents, surgical/dental devices, catheters, syringes, needles, blood collection tubes and bags, disposable contact lenses, prostheses, implants, pacemakers, pacemaker leads, heart valves, pulse generators, cardiac defibrillators, cardioverter defibrillators, spinal stimulators, brain and nerve stimulators, introducers, chemical sensors, artificial joints, skin/vascular grafts, bandages and dressings, chemical and physiological electrodes/sensors, personal hygiene and contraceptive items. Optionally, the amine-functionalized polysilane coatings of the present invention can also be used to bind and selectively deliver drugs, prodrugs, nucleotides, oligonucleotides, polynucleotides, amino acids, proteins, saccharides as well as fix tissue slices/specimens for histological or pathological examination, and the like, according to methods known in the art.

The following examples further illustrate the invention but, of course, should not be construed as in any way limiting its scope.

### EXAMPLE 1

This example illustrates the preparation of a diazeniumdiolated substituted ammonium 1-aminopropylsiloxane-5-PEI-2,4-dinitrobenzene-coated stainless steel coupon.

A 1×1 cm sheet of medical-grade stainless steel was placed in a 13×100 mm test tube containing a neat solution of 3-aminopropyltrimethoxysilane. After 3 minutes of exposure, excess silane reagent was removed. The coupon was washed with methanol and diethyl ether, and dried under nitrogen for several minutes until the residual solvent had completely evaporated. The test tube containing the coupon was placed in an oven at 110° C. for 15 minutes. The test tube was removed from the oven and allowed to cool to room temperature.

The coupon was transferred to a new test tube and 2 mL of a tetrahydrofuran (THE) solution containing 40 mg of 1,5-difluoro-2,4-dinitrobenzene and 20 mg of anhydrous potassium carbonate was added. Using a hot air dryer, the test tube was then carefully heated until the solution began to boil whereupon it was immediately placed in a metal test tube rack and allowed to slowly cool to room temperature. The solution was removed, and the coupon was washed with an additional 20 mL of THF.

The cross-linked derivatized medical coupon was treated with 2 mL of THF containing a slurry of 40 mg of linear polyethylenimine (“PEI” MW=25,000 g/mol). The test tube was heated until the THF began to boil and was allowed to cool to room temperature. Excess solvent was removed from the tube, and the coupon was washed with 20 mL each of THF and diethyl ether. The coupon was dried under nitrogen and transferred to a new test tube. Three (3) mL of acetonitrile were added, and the tube was placed in a Part® hydrogenation pressure vessel. Oxygen was removed from the vessel using repeated cycles of pressurization/depressurization with nitrogen gas. This was followed by introduction of 276 kPa (40 psi) of NO. The tube containing the coupon was left overnight in the NO apparatus. The acetonitrile was decanted and the coupon was washed with 20 mL of diethyl ether and dried under nitrogen.

The diazeniumdiolated coupon was immersed in 0.1 M phosphate buffer, pH 7.4 at 37° C., whereupon chemiluminescence-detectable NO was evolved over an approximately 4 day period of analysis. The total NO release was measured at 1704 pmoles/mg of polymer.

### EXAMPLE 2

This example illustrates the preparation of a 1-aminopropylsiloxane-5-methoxymethyl-protected monodiazeniumdiolate of piperazine-2,4-dinitrobenzene-coated stainless steel coupon.

Per the method outlined above, 100 mg of a methoxymethyl-protected monodiazeniumdiolate of piperazine derivative was coupled to the surface of a 1-aminopropylsiloxane-5-fluoro-2,4-dinitrobenzene-coated metal coupon. When immersed in a 1.0 M phosphate buffer, pH 7.4 at 37° C., chemiluminescence-detectable NO was evolved at a negligible initial rate. After 15 minutes, 1 mL of a 25% sulfuric acid solution was added, whereupon 551 pmoles of NO was detected over a period of 2.26 h.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for preparing a nitric oxide-releasing substrate comprising:
(a) contacting an amine-functionalized silane residue with a substrate;
(b) contacting the amine-functionalized silane residue with a cross-linking agent; and
(c) contacting at least one nitric oxide-releasing functional group with the cross-linked amine-functionalized silane residue.

2. The method according to claim 1, wherein the substrate comprises a metal, glass, plastic, rubber, or ceramic.

3.-13. (canceled)

14. The method according to claim 1, wherein the amine-functionalized silane is selected from the group consisting of 3-aminopropyltrimethoxysilane, 3-aminopropyltriethoxysilane, 3-aminopropyldimethoxysilane, N-(3-acryloxy-2-hydroxypropyl)-3-amino-propyltriethoxysilane, N-2-(aminoethyl)-3-aminopropyltris(2-ethyl-hexoxy)silane, 3-(m-aminophenoxy)propyltrimethoxysilane, 3-(1-aminopropoxy)-3,3-dimethyl-1-propenyl-trimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylchlorosilane, bis(dimethylamino)methylmethoxysilane, bis(dimethylamino)phenylchlorosilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetriamine, trimethoxysilylpropylpentaethylenehexamine, triethoxysilyloctyldiethylenetriamine, triisopropoxysilylpentaethylenehexamine, n-trimethoxysilylpropyl-N,N,N-trimethylammonium chloride, 3-aminopropymethyldiethoxysilane, 2-(perfluorooctyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-N′-[3-(dimethoxymethylsilyl)propyl]-1,2-ethanediamine, amine-functionalized polydimethylsiloxane copolymer, and bis-aminosilane.

15. The method according to claim 1, wherein the cross-linking agent comprises a dihalogenated alkyl or a dihalogenated aryl, wherein the cross-linking agent is optionally substituted with a substituent selected from the group consisting of an alkyl, a cycloalkyl, hydroxyl, nitro, a halogen, cyano, and combinations thereof.

16. (canceled)

17. The method according to claim 15, wherein the cross-linking agent is 1,4-dibromoethane or 1,5-difluoro-2,4-dinitrobenzene.

18. The method according to claim 1, wherein the nitric oxide-releasing functional group comprises a nitric oxide-releasing functional group that is an O²-protected diazeniumdiolate of an amine-functionalized silane.

19. (canceled)

20. A method for preparing a nitric oxide-releasing metallic substrate comprising:
(a) contacting an aminopropyltrimethoxysilane solution with a substrate;
(b) contacting the aminopropyltrimethoxysilane with a cross-linking agent selected from the group consisting of 1,4-dibromoethane and 1,5-difluoro-2,4-dinitrobenzene; and
(c) contacting either
(i) an O²-protected diazeniumdiolate with the cross-linked aminopropyl trimethoxysilane or
(ii) an amine-derived residue selected from the group consisting of diethylenetriamine, pentaethylenehexamine, high molecular weight linear/branched polyethylenimines, amine-functionalized divinylbenzene, and piperazine with the cross-linked aminopropyltrimethoxysilane, and contacting the amine-derived residue with nitric oxide gas.

21. (canceled)

22. A method for preparing a nitric oxide-releasing substrate comprising:
(a) contacting an amine-functionalized silane residue solution with a substrate;
(b) contacting the amine-functionalized silane residue with a cross-linking agent;
(c) contacting at least one nucleophilic residue with the cross-linked amine-functionalized silane residue; and
(d) contacting the nucleophilic residue with nitric oxide gas.

23. The method according to claim 22, wherein the nucleophilic residue is an amine-derived residue.

24. The method according to claim 23, wherein the amine-derived residue is selected from the group consisting of diethylenetriamine, pentaethylenehexamine, high molecular weight linear/branched polyethylenimines, amine-functionalized divinylbenzene, piperazine, and combinations thereof.

25. The method according to claim 22, further comprising: after step (c), contacting the nucleophilic residue with a cross-linking agent, and contacting at least one additional nucleophilic residue with the cross-linked nucleophilic residue.

26. The method according to claim 22, further comprising: prior to step (d), treating the substrate having the cross-linked amine-functionalized silane residue with a biocompatible topcoat.

27. The method according to claim 26, wherein the biocompatible topcoat is a lubricous hydrogel selected from the group consisting of homo- and heteropolyethers, polyols, polyureas, polylactones albumin-, heparin-, and polyphosphorylcholine-functionalized polymers, and combinations thereof.

28.-31. (canceled)

32. A medical device for delivering nitric oxide in a therapeutic concentration, the device comprising a substrate having nitric oxide bound thereto through a diazeniumdiolated nucleophile bonded to a silane intermediate, the silane intermediate being amine-functionalized and cross-linked.

33. The medical device according to claim 32, wherein the device comprises metal.

34. (canceled)

35. The medical device according to claim 32, wherein the silane intermediate is selected from the group consisting of 3-aminopropyltrimethoxysilane, 3-aminopropyltriethoxysilane, 3-aminopropyldimethoxysilane, N-(3-acryloxy-2-hydroxypropyl)-3-amino-propyltriethoxysilane, N-2-(aminoethyl)-3-aminopropyltris(2-ethyl-hexoxy)silane, 3-(m-aminophenoxy)propyltrimethoxysilane, 3-(1-aminopropoxy)-3,3-dimethyl-1-propenyl-trimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylchlorosilane, bis(dimethylamino)methylmethoxysilane, bis(dimethylamino)phenylchlorosilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetriame trimethoxysilylpropylpentaethylenehexamine, triethoxysilyloctyldiethylenetriamine, triisopropoxysilylpentaethylenehexamine, n-trimethoxysilylpropyl-N,N,N-trimethylammonium chloride, 3-aminopropylmethyldiethoxysilane, 2-(perfluorooctyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-]N′-[3-(dimethoxymethylsilyl)propyl]-1,2-ethanediamine, amine-functionalized polydimethylsiloxane copolymer, and bis-aminosilane.

36. The medical device according to claim 32, wherein the silane intermediate is cross-linked using a dihalogenated alkyl or a dihalogenated aryl that is optionally substituted with a substituent selected from the group consisting of an alkyl, a cycloalkyl, hydroxyl, nitro, a halogen, and cyano.

37. (canceled)

38. (canceled)

39. The medical device according to claim 36, wherein the cross-linking agent is 1,4-dibromoethane or 1,5-difluoro-2,4-dinitrobenzene.

40. The medical device according to claim 32, wherein the diazeniumdiolated nucleophile comprises a nitric oxide-releasing functional group that is an O²-protected diazeniumdiolate of an amine-functionalized silane.

41. The medical device according to claim 32, wherein the medical device is selected from the group consisting of an arterial stent, guide wire, catheter, trocar needle, bone anchor, bone screw, protective plating, hip and joint implant, electrical lead, biosensor, and a probe.

42. An arterial stent for delivering nitric oxide in a therapeutic concentration, the device comprising a metallic substrate having nitric oxide releasably bound thereto through an O²-protected diazeniumdiolate bonded to an aminopropyltrimethoxysilane, the aminopropyltrimethoxysilane being cross-linked by a cross-linking agent selected from the group consisting of 1,4-dibromoethane and 1,5-difluoro-2,4-dinitrobenzene.

43. A method for preparing a nitric oxide-releasing substrate comprising:
(a) contacting a non- or weakly-nucleophilic silane residue with a substrate;
(b) contacting the silane residue with a cross-linking agent; and
(c) contacting nitric oxide with the cross-linked silane residue.
